(19) United States
(12) Patent Application Publication
(10) Pub. No.: US 2008/0200498 A1
(43) Pub. Date: Aug. 21, 2008

(54) Pharmaceutical Composition For The Treatment Of Disorders Of Sexual Desire

(76) Inventors: Angelo Ceci, Mittelbiberach (DE); Klaus Mendla, Biberach (DE)

Correspondence Address: MICHAEL P. MORRIS;BOEHRINGER INGELHEIM USA CORPORATION, 900 RIDGEBURY ROAD, P. O. BOX 368, RIDGEFIELD, CT, 06877-0368, US

(21) Appl. No.: 11/994,741

(22) PCT Filed: Jul. 6, 2006
(86) PCT No.: PCT/EP2006/063991

§ 371 Date: Apr. 17, 2008

(30) Foreign Application Priority Data
Jul. 12, 2005 (EP) 05015110.9

(51) Int. Cl. A61K 31/46; A61P 15/12; A61P 15/02; A61P 15/10

(52) U.S. Cl. 514/304

(57) ABSTRACT

The invention relates to the use of a monoamine neurotransmitter re-uptake inhibitor comprising a 2,3-disubstituted tropane moiety, or a tautomer, a pharmaceutically acceptable salt, solvate, or physiologically functional derivative thereof for the manufacture of a medicament for the for the treatment of sexual desire disorders.

The invention relates to the use of a monoamine neurotransmitter re-uptake inhibitor comprising a 2,3-disubstituted tropane moiety, or a tautomer, a pharmaceutically acceptable salt, solvate, or physiologically functional derivative thereof for the manufacture of a medicament for the treatment of disorders of sexual desire.

### BACKGROUND INFORMATION

The majority of people who suffer from sexual dysfunction are afraid to speak about their complaints and the options of treatment with their physicians. Usually, sexual dysfunction is associated with male impotence which in consequence raise the uneasiness for women to speak about this issue although it is estimated that alone in America more than 30% of women suffer from sexual dysfunction. Reasons for sexual dysfunction may be drug-related, organ-related, hormone-related or they are psychologically based. Despite the need to find medicament solutions to treat such disorders in particular in women, there are only few results. Female sexual disorders include hypoactive sexual desire disorders, sexual arousal disorders, anorgasmy and sexual pain disorders

The International patent applications WO 93/09814 and WO 97/30997 disclose tropane derivatives, which are monoamine neurotransmitter re-uptake inhibitors. However, there is no indication that such compounds may develop properties to treat hyposexual desire disorders.

### SUMMARY OF THE INVENTION

It has been found that 2,3-disubstituted tropanes may display sexual desire enhancing properties

The objective of the invention is to provide a medicament to treat sexual desire disorders, in particular in women.

Accordingly, the present invention relates to the use of a monoamine neurotransmitter re-uptake inhibitor comprising a 2,3-disubstituted tropane moiety, or a tautomer, a pharmaceutically acceptable salt, solvate, or physiologically functional derivative thereof for the manufacture of a medicament for the treatment of female sexual desire disorders, in particular hyposexual desire disorder (HSDD), loss of sexual desire, lack of sexual desire, decreased sexual desire, inhibited sexual desire.

The beneficial effects of the compounds of the present invention can be observed regardless of whether the disturbance existed lifelong or was acquired, and independent of etiologic origin (organic—both, physically and drug induced-, psychogen, a combination of organic—both, physically and drug induced-, and psychogen, or unknown).

### DETAILED DESCRIPTION OF THE INVENTION

As a rule the monoamine neurotransmitter re-uptake inhibitor comprising a 2,3-disubstituted tropane moiety are those which are disclosed by International patent applications WO 93/09814 and WO 97/30997.

For use according to the invention, it is preferable to use a compound of the general formula (I)

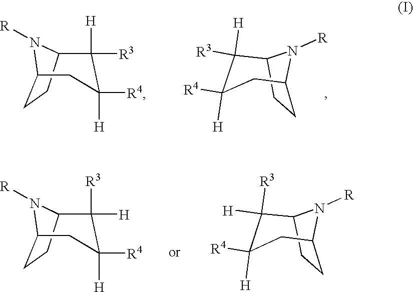

or a pharmaceutical acceptable addition salt thereof or the N-oxide thereof, wherein

R is hydrogen, alkyl, alkenyl, alkynyl, cycloalkyl, cycloalkylalkyl or 2-hydroxyethyl;
R³ is CH₂—X—R′, wherein X is O, S, or NR″; R″ is hydrogen or alkyl; and R′ is alkyl, alkenyl, alkynyl, cycloalkyl, cycloalkylalkyl, or —CO-alkyl;
R⁴ is phenyl which may be substituted one or more times with substituents selected from the group consisting of halogen, CF₃, CN, alkoxy, cycloalkoxy alkyl, cycloalkyl, alkenyl, alkynyl, amino, nitro, and heteroaryl and aryl;
   3,4-methylenedioxyphenyl;
   benzyl which may be substituted one or more times with substituents selected from the group consisting of halogen, CF₃, CN, alkoxy, cycloalkoxy alkyl, cycloalkyl, alkenyl, alkynyl, amino, nitro, and heteroaryl and aryl;
   heteroaryl, which may be substituted one or more times with substituents selected from the group consisting of halogen, CF₃, CN, alkoxy, cycloalkoxy alkyl, cycloalkyl, alkenyl, alkynyl, amino, nitro, and heteroaryl and aryl;
   naphtyl, which may be substituted one or more times with substituents selected from the group consisting of halogen, CF₃, CN, alkoxy, cycloalkoxy alkyl, cycloalkyl, alkenyl, alkynyl, amino, nitro, and heteroaryl and aryl.

In a special embodiment of the compound of general formula I, R³ is 1,2,4-oxadiazol-3-yl which may by substituted in the 5 position with alkyl, cycloalkyl, or cycloalkylalkyl; phenyl which may be substituted one or more times with substituents selected from the group consisting of halogen, CF₃, CN, alkoxy, alkyl, alkenyl, alkynyl, amino, nitro, and heteroaryl; phenyl; or benzyl which may be substituted one or more times with substituents selected from the group consisting of halogen, CF₃, CN, alkoxy, alkyl, alkenyl, alkynyl, amino, nitro, and heteroaryl; or 1,2,4-oxadiazol-5-yl which may by substituted in the 3 position with alkyl, cycloalkyl, or cycloalkylalkyl; phenyl which may be substituted one or more times with substituents selected from the group consisting of halogen, CF₃, CN, alkoxy, alkyl, alkenyl, alkynyl, amino, nitro, and heteroaryl; phenyl; benzyl which may be substituted one or more times with substituents selected from the group consisting of halogen, CF₃, CN, alkoxy, alkyl, alkenyl, alkynyl, amino, nitro, and heteroaryl; pyridyl which may be substituted one or more times with substituents selected from the group consisting of halogen, CF₃, CN, alkoxy, alkyl, alkenyl, alkynyl, amino, nitro and heteroaryl; or thienyl which may be substituted one or more times with substituents selected from the group consisting of halogen, CF₃, CN, alkoxy, alkyl, alkenyl, alkynyl, amino, nitro and heteroaryl.

In a further special embodiment of the compound of general formula (I), R³ is .CH₂—X—R′, wherein X is O, S, or NR″; wherein R″ is hydrogen or alkyl; and

R′ is alkyl, alkenyl, alkynyl, cycloalkyl, cycloalkylalkyl, or —CO-alkyl.

In a still further embodiment of the compound of general formula (I), R³ is CH═NOR′; wherein R′ is hydrogen; alkyl, cycloalkyl, cycloalkylalkyl, alkenyl, alkynyl or aryl; all of which may be substituted with —COOH; —COO-alkyl; —COO-cycloalkyl; or phenyl which may be substituted one or more times with substituents selected from the group consisting of halogen, CF₃, CN, alkyl, cycloalkyl, alkoxy, cycloalkoxy, alkenyl, alkynyl, amino, and nitro.

In a further special embodiment of the compound of general formula (I), R⁴ is phenyl, which is substituted once or twice with substituents selected from the group consisting of halogen, CF₃, CN, alkoxy, cycloalkoxy, alkyl, cycloalkyl, alkenyl, alkynyl, amino, nitro, and heteroaryl.

In a more special embodiment, R⁴ is phenyl substituted once or twice with chlorine.

In a further special embodiment, the tropane derivative having dopamine reuptake inhibitor activity is a (1R,2R,3S)-2,3-disubstituted tropane derivative of formula I.

In a still further embodiment, the tropane derivative having dopamine reuptake inhibitory activity is a compound of general formula I wherein R³ is —CH₂—X—R′, wherein X is O or S, and R′ is methyl, ethyl, propyl, or cyclopropylmethyl; —CH═NOR′; wherein R′ is hydrogen or alkyl, or 1,2,4-oxadiazol-5-yl which may by substituted in the 3 position with alkyl.

In a still further embodiment, the tropane derivative having dopamine reuptake inhibitory activity is a compound of general formula I wherein R is hydrogen, methyl, ethyl or propyl.

In a still further embodiment, the tropane derivative having dopamine reuptake inhibitory activity is a compound of general formula I wherein R⁴ is 3,4-dichlorophenyl.

Preferably those monoamine neurotransmitter re-uptake inhibitor comprising a 2,3-disubstituted tropane moiety are compounds of formula (II)

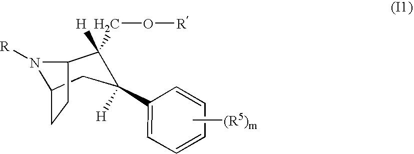

wherein
R represents a hydrogen atom or a C₁₋₆ alkyl group, preferably a hydrogen atom, a methyl or an ethyl group;
R⁵ each independently represents a halogen atom or a CF₃ or cyano group, preferably a fluorine, chlorine or bromine atom;
R′ represents a hydrogen atom or a C₁₋₆ alkyl or C₃₋₆-cycloalkyl-C₁₋₃-alkyl group, preferably a methyl, ethyl or n-propyl group; and
m is 0 or an integer from 1 to 3, preferably 1 or 2;
or a tautomer, a pharmaceutically acceptable salt, solvate, or physiologically functional derivative thereof.

As used herein, the expression “C₁₋₆ alkyl” includes methyl and ethyl groups, and straight-chained and branched propyl, butyl, pentyl and hexyl groups. Particular alkyl groups are methyl, ethyl, n-propyl, isopropyl and t-butyl.

The expression “C₃₋₆ cycloalkyl” as used herein includes cyclic propyl, butyl, pentyl and hexyl groups such as cyclopropyl and cyclohexyl.

The term “halogen” as used herein includes fluorine, chlorine, bromine and iodine, of which fluorine and chlorine are preferred.

The term “physiologically functional derivative” as used herein includes derivatives obtained from the compound of formula (I) under physiological conditions, these are for example N-oxides, which are formed under oxidative conditions.

The term “pharmaceutically acceptable acid addition salt” as used herein includes those salts which are selected from among the acid addition salts formed with hydrochloric acid, hydrobromic acid, sulphuric acid, phosphoric acid, methanesulphonic acid, acetic acid, fumaric acid, succinic acid, lactic acid, citric acid, tartaric acid and maleic acid, the salts obtained from hydrochloric acid, hydrobromic acid, sulphuric acid, phosphoric acid and acetic acid being particularly preferred. The salts of citric acid are of particular significance.

In a special embodiment, the tropane derivative having dopamine reuptake inhibitor activity is a compound of the general formula (I) selected from:
(1R,2R,3S)-2-(3-Cyclopropyl-1,2,4-oxadiazol-5-yl)-3-(4-fluorophenyl)tropane;
(1R,2R,3S)-2-(3-Phenyl-1,2,4-oxadiazol-5-yl)-3-(4-fluorophenyl)tropane;
(1R,2R,3S)-2-(3-Phenyl-1,2,4-oxadiazol-5-yl)-3-(4-methylphenyl)-tropane;
(1R,2R,3S)-2-(3-Benzyl-1,2,4-oxadiazol-5-yl)-3-(4-fluorophenyl)tropane;
(1R,2R,3S)-2-(3-(4-Phenyl-phenyl)-1,2,4-oxadiazol-5-yl)-3-(4-fluorophenyl)tropane;
(1R,2R,3S)-2-(3-Phenyl-1,2,4-oxadiazol-5-yl)-3-(2-naphthyl)tropane;
(1R,2R,3S)-3-(3,4-Dichlorophenyl)tropane-2-aldoxime;
(1R,2R,3S)-3-(3,4-Dichlorophenyl)-tropane-2-O-methyl-aldoxime;
(1R,2R,3S)-3-(3,4-Dichlorophenyl)tropane-2-O-benzyl-aldoxime;
(1R,2R,3S)-3-(3,4-Dichlorophenyl)tropane-2-O-ethoxycarbonylmethyl-aldoxime;
(1R,2R,3S)-3-(3,4-Dichlorophenyl)tropane-2-O-methoxycarbonylmethyl-aldoxime;
(1R,2R,3S)-3-(3,4-Dichlorophenyl)tropane-2-O-(1-ethoxycarbonyl-1,1-dimethyl-methyl)-aldoxime;
(1R,2R,3S)-3-(3,4-Dichlorophenyl)tropane-2-O-carboxymethyl-2-aldoxime;
(1R,2R,3S)—N-Normethyl-3-(3,4-dichlorophenyl)tropane-2-O-methyl-aldoxime;
(1R,2R,3S)—N-Normethyl-3-(3,4-dichlorophenyl)tropane-2-O-benzyl-aldoxime;
(1R,2R,3S)-3-(4-Methylphenyl)tropane-2-O-methyl-aldoxime;
(1R,2R,3S)-3-(3,4-Dichlorophenyl)tropane-2-O-(1,1-dimethylethyl)-aldoxime;
(1R,2R,3S)-3-(4-Chlorophenyl)tropane-2-O-aldoxime;
(1R,2R,3S)-3-(4-Chlorophenyl)tropane-2-O-methylaldoxime hydrochloride;
(1R,2R,3S)-3-(4-Chlorophenyl)tropane-2-O-methoxycarbonylmethyl-aldoxime;
(1R,2R,3S)-3-(3,4-Dichlorophenyl)tropane-2-O— (2-propynyl)-aldoxime;
(1R,2R,3S)-3-(3,4-Dichlorophenyl)tropane-2-O-(2-methylpropyl)-aldoxime;
(1R,2R,3S)-3-(3,4-Dichlorophenyl)tropane-2-O-cyclopropylmethyl-aldoxime;
(1R,2R,3S)-3-(3,4-Dichlorophenyl)tropane-2-O-ethyl-aldoxime;
(1R,2R,3S)-2-Methoxymethyl-3-(3,4-dichlorophenyl)-tropane;
(1R,2R,3S)-2-Isopropoxymethyl-3-(3,4-dichlorophenyl)-tropane;
(1R,2R,3S)-2-Ethoxymethyl-3-(3,4-dichlorophenyl)-tropane;
(1R,2R,3S)-2-Ethoxymethyl-3-(3,4-dichlorophenyl)-nortropane;
(1R,2R,3S)-2-Cyclopropylmethyloxymethyl-3-(3,4-dichlorophenyl)-tropane;
(1R,2R,3S)-2-Methoxymethyl-3-(4-chlorophenyl)-tropane;
(1R,2R,3S)—N-Normethyl-2-methoxymethyl-3-(4-chlorophenyl)-tropane;
(1R,2R,3S)-2-Ethoxymethyl-3-(4-chlorophenyl)-tropane;
(1R,2R,3S)—N-Normethyl-2-methoxymethyl-3-(3,4-dichlorophenyl)-tropane;
(1R,2R,3S)—N-Normethyl-2-ethoxymethyl-3-(3,4-dichlorophenyl)-tropane;
(1R,2R,3S)—N-Normethyl-2-ethoxymethyl-3-(4-chlorophenyl)-tropane;
(1R,2R,3S)—N-Normethyl-2-cyclopropylmethyloxymethyl-3-(4-chlorophenyl)-tropane;
(1R,2R,3S)-2-Cyclopropylmethyloxymethyl-3-(4-chlorophenyl)-tropane;
(1R,2R,3S)-2-Ethylthiomethyl-3-(3,4-dichlorophenyl)-tropane;
(1R,2R,3S)-2-Hydroxymethyl-3-(4-fluorophenyl)tropane;
(1R,2R,3S)-2-Hydroxymethyl-3-(3,4-dichlorophenyl)tropane;
(1R,2R,3S)—N-Normethyl-N-(tert-butoxycarbonyl)-2-hydroxymethyl-3-(3,4-dichlorophenyl)tropane;
(1R,2R,3S)-2-Hydroxymethyl-3-(4-chlorophenyl)tropane;
(1R,2R,3S)-2-(3-(2-Furanyl)-1,2,4-oxadiazol-5-yl)-3-(3,4-dichlorophenyl)-tropane;
(1R,2R,3S)-2-(3-(3-Pyridyl)-1,2,4-oxadiazol-5-yl)-3-(3,4-dichlorophenyl)-tropane;
(1R,2R,3S)—N-Normethyl-N-allyl-2-(3-(4-pyridyl)-1,2,4-oxadiazol-5-yl)-3-(3,4-dichlorophenyl)-tropane;
(1R,2R,3S)—N-Normethyl-N-ethyl-2-(3-(4-pyridyl)-1,2,4-oxadiazol-5-yl)-3-(3,4-dichlorophenyl)-tropane;
(1R,2R,3S)—N-Normethyl-N-(2-hydroxyethyl)-2-(3-(4-pyridyl)-1,2,4-oxadiazol-5-yl)-3-(3,4-dichlorophenyl)-tropane;
(1R,2R,3S)—N-Normethyl-2-(3-(4-pyridyl)-1,2,4-oxadiazol-5-yl)-3-(3,4-dichlorophenyl)-tropane;
(1R,2R,3S)—N-Normethyl-N-allyl-2-(3-(3-pyridyl)-1,2,4-oxadiazol-5-yl)-3-(3,4-dichlorophenyl)-tropane;
(1R,2R,3S)—N-Normethyl-N-allyl-2-(3-(2-pyridyl)-1,2,4-oxadiazol-5-yl)-3-(3,4-dichlorophenyl)-tropane;
(1R,2R,3S)-2-(3-(2-Thienyl)-1,2,4-oxadiazol-5-yl)-3-(4-chlorophenyl)-tropane;
(1R,2R,3S)-2-(3-(2-Thienyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(3,4-dichlorophenyl)-tropane;
(1R,2R,3S)-2-(3-(2-Pyridyl)-1,2,4-oxadiazol-5-yl)-3-(3,4-dichlorophenyl)-tropane;
(1R,2R,3S)-2-(3-(4-Pyridyl)-1,2,4-oxadiazol-5-yl)-3-(4-chlorophenyl)-tropane;
(1R,2R,3S)-2-(3-(3-Pyridyl)-1,2,4-oxadiazol-5-yl)-3-(4-chlorophenyl)-tropane;
(1R,2R,3S)-2-(3-2-Pyridyl)-1,2,4-oxadiazol-5-yl)-3-(4-chlorophenyl)-tropane;
(1R,2R,3S)-2-(3-Phenyl-1,2,4-oxadiazol-5-yl)-3-(4-fluorophenyl)-tropane;
(1R,2R,3S)-2-(3-Phenyl-1,2,4-oxadiazol-5-yl)-3-(4-methylphenyl)-tropane;
(1R,2R,3S)-2-(3-Benzyl-1,2,4-oxadiazol-5-yl)-3-(4-fluorophenyl)-tropane;
(1R,2R,3S)-2-(3-(4-Phenylphenyl)-1,2,4-oxadiazol-5-yl)-3-(4-fluorophenyl)-tropane;
(1R,2R,3S)-2-(3-Phenyl-1,2,4-oxadiazol-5-yl)-3-(2-naphthyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(4-fluorophenyl)-tropane;
(1R,2R,3S)-2-(4-Chlorophenoxy-methyl)-3-(3,4-dichlorophenyl)-tropane;
(1R,2R,3S)-2-(4-Chlorophenoxy-methyl)-3-(4-methylphenyl)-tropane;
(1R,2R,3S)-2-(4-Benzoyloxy-methyl)-3-(4-fluorophenyl)-tropane;
(1R,2R,3S)-2-Carbomethoxy-3-(2-naphthyl)-tropane;
(1R,2R,3S)-2-Carbomethoxy-3-(3,4-dichlorophenyl)-tropane;
(1R,2R,3S)-2-Carbomethoxy-3-benzyl-tropane;
(1R,2R,3S)-2-Carbomethoxy-3-(4-chlorophenyl)-tropane;
(1R,2R,3S)-2-Carbomethoxy-3-(4-methylphenyl)-tropane;
(1R,2R,3S)-2-Carbomethoxy-3-(1-naphthyl)-tropane;
(1R,2R,3S)-2-Carbomethoxy-3-(4-phenylphenyl)-tropane;
(1R,2R,3S)-2-Carbomethoxy-3-(4-t-butyl-phenyl)-tropane;
(1R,2R,3S)-2-(4-Fluoro-benzoyl)-3-(4-fluorophenyl)-tropane; or a pharmaceutically acceptable addition salt thereof.

Most preferred are the compounds of formulae (IA) and (IB)

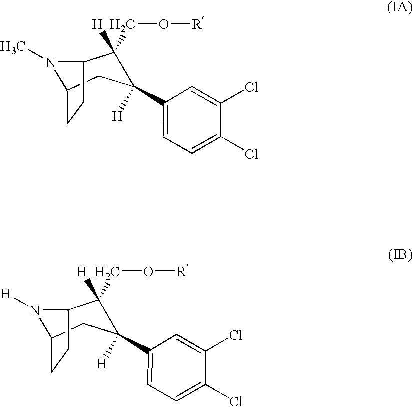

with R′ being a C₁₋₃ alkyl, preferably methyl, ethyl, propyl. The compounds are coded COMPOUND IA, and COMPOUND IB.

It is particularly preferable to use the monoamine neurotransmitter re-uptake inhibitor comprising a 2,3-disubstituted tropane moiety to prepare a pharmaceutical composition for the treatment of HSDD and loss of sexual desire.

Preferably the patients are female adults of any race, in particular aged 45 and above, most preferred aged 60 and above.

It is preferred to provide an orally available pharmaceutical composition, most preferred are tablets and the like.

But beside this route of administration transdermal, inhalative, intrathecal, parenteral or transvaginal applications are possible, e.g. vaginal suppositories.

Suitable preparations include, for example, tablets, particularly slow release tablets, capsules, suppositories, solutions, syrups, emulsions, dispersible powders, implants or plasters, most preferably micronal plasters. Tablets may be obtained, for example, by mixing the active substance or substances with known excipients, e.g. inert diluents such as calcium carbonate, calcium phosphate or lactose, disintegrants such as maize starch or alginic acid, binders such as starch or gelatine, lubricants such as magnesium stearate or talc, and/or agents for obtaining delayed release such as carboxymethylcellulose, cellulose acetate phthalate, or polyvinylacetate. The tablets may also consist of several layers.

The monoamine neurotransmitter re-uptake inhibitors of formulae IA and IB which are preferably used within the scope of the present invention may optionally (and this is preferred) be used in the form of their pharmacologically acceptable acid addition salts, and/or in the form of the hydrates and solvates.

By the pharmaceutically acceptable acid addition salts of the dopamine monoamine neurotransmitter re-uptake inhibitor of formula I are meant, according to the invention, those salts which are selected from among the salts of hydrochloric acid, hydrobromic acid, sulphuric acid, phosphoric acid, methanesulphonic acid, acetic acid, fumaric acid, succinic acid, lactic acid, citric acid, tartaric acid and maleic acid, the salts of hydrochloric acid, hydrobromic acid, sulphuric acid, phosphoric acid and acetic acid being particularly preferred. The salts of citric acid are of particular significance.

In the case of formulae IA and IB, the use of which is particularly preferred according to the invention, the citrate is of particular importance. For transdermal administration it is preferable to use the base of formula I.

The monoamine neurotransmitter re-uptake inhibitor comprising a 2,3-disubstituted tropane moiety, preferably the compounds of formula I, most preferably of formula IA and IB, which may be used according to the invention may optionally be used in conjunction with other active substances. Preferred combination partners are compounds selected from the categories of the D₁₋, D₂₋, D₃₋ or D₄₋ agonists or a pharmaceutically acceptable salt, solvate, or physiologically functional derivative thereof, preferably selected from among group consisting of, adrogolide, A-86929, Rotigotine, NeurVex, nolomirole, pramipexole, talipexol, CHF 1512, (−)-stepholidine, DAR-201, diacrin/Genzyme, bromocriptine, bupropion, LEK-8829, BAM-1110, AIT-203, terguride, aripiprazole, OPC-4392, GMC-1111, PD-148903, apomorphine HCl, PD-89211, PD-158771, cabergoline, sumanirole, PNU-14277E, POL-255, dihydrexidine, GBR-12783, quinagolide HCl, (R)-bupropion, S-32504, S-33592, SKF-80723, SKF-83959, fenoldopam, Flibanserin, ropinirole, SKF-82958, SKF-77434, DU 127090, SLV-308, SLV 318, NeuroCRIB, SP-1037C, spheramine, gallotrank, preclamol, DAB-452, YM-435, BP-897, ProSavin, etilevodopa, P63, A 68930, A 77636, alaptide, alentemol, CI 1007; PD 143188, BLSI, JA 116a; JA 116, melevodopa; levodopa methyl; CHF 1301; NSC 295453; levomet, MR 708, PD 128483, RD 211, SKF 38393, SKF 81297, U 86170F, U 91356A, WAY 124486, Z 15040, silbutramine, orlistat, amfepramon-HCl and ephedrine.

The dosage of the monoamine neurotransmitter re-uptake inhibitors according to the invention is naturally adapted to the severity of the symptoms to be treated. For example, without restricting the subject matter of the present invention thereto, some possible dosages especially for the compounds of formula IA and IB which are particularly preferred according to the invention will now be given. This may be used in dosages of about 0.05 to 10 mg, preferably about 0.1 to 2.0 mg, in particular about 0.125 to 1.0 mg daily or 0.1 to 5 mg once weekly. These dosages are based on the compound of formula IA in the form of its free base. Based on the salt form which is preferably used, namely the citrate, the above mentioned dosages correspond to about 0.08 to 16 mg, preferably 0.16 to 2.38 mg, in particular about 0.20 to 1.58 of the compound of formula IA citrate per day.

One possible dosing method, which is described solely as an illustrative example, is described hereinafter (based on the compound of formula IA in the form of its free base): with or without individual dosage titration at weekly intervals depending on the activity and tolerance levels.

Some examples of pharmaceutical preparations which may be used preferably for formula IA and IB which may be used according to the invention are given below. These are intended solely as illustrations by way of example without restricting the subject matter of the invention thereto.

Tablet 1:

| Ingredients: | mg |
|---|---|
| COMPOUND IA | 1.00 |
| Mannitol | 121.50 |
| Maize starch | 79.85 |
| Highly dispersed silicon dioxide, anhydrous | 2.30 |
| Polyvidon K25 | 2.35 |
| Magnesium stearate | 3.00 |
| Total | 210.00 |

Tablet 2:

| Ingredients: | mg |
|---|---|
| COMPOUND IA | 0.5 |
| Mannitol | 122.0 |
| Maize starch, dried | 61.8 |
| Maize starch | 18.0 |
| Highly dispersed silicon dioxide, anhydrous | 2.4 |
| Polyvidon K25 | 2.3 |
| Magnesium stearate | 3.0 |
| Total | 210.0 |

Tablet 3:

| Ingredients: | mg |
|---|---|
| COMPOUND IA | 0.25 |
| Mannitol | 61.00 |
| Maize starch | 39.90 |
| Highly dispersed silicon dioxide, anhydrous | 1.20 |
| Polyvidon K25 | 1.15 |
| Magnesium stearate | 1.5 |
| Total | 105.00 |

Tablet 4:

| Ingredients: | mg |
|---|---|
| COMPOUND IA | 0.125 |
| Mannitol | 49.455 |
| Maize starch, dried | 25.010 |
| Maize starch | 7.300 |
| Highly dispersed silicon dioxide, anhydrous | 0.940 |
| Polyvidon K25 | 0.940 |
| Magnesium stearate | 1.230 |
| Total | 85.000 |

### Solution for Injection:

| | | |
|---|---|---|
| COMPOUND IA | 0.3 | mg |
| sodium chloride | 0.8 | mg |
| benzalkonium chloride | 0.01 | mg |
| water for injections | ad 100 | ml |

COMPOUND IA, includes any of the three possibilities, the methyl-, ethyl and propylether.

Instead of COMPOUND IA also COMPOUND IB may be used.

1. A method for the treatment a disorder of sexual desire, in particular in a women, the method comprising the step of administering a monoamine neurotransmitter re-uptake inhibitor comprising a 2,3-disubstituted tropane moiety to a patient in need thereof.

2. The method according to claim 1, wherein the disorder of sexual desire is selected from the group consisting of Hypoactive Sexual Desire Disorder, loss of sexual desire, lack of sexual desire, decreased sexual desire, inhibited sexual desire, loss of libido, libido disturbance, and frigidity.

3-4. (canceled)

5. The method according to claim 1, wherein said monoamine neurotransmitter re-uptake inhibitor comprising a 2,3-disubstituted tropane moiety is a compound of formula (I) 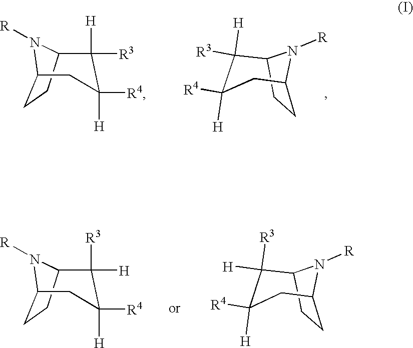
or a pharmaceutical acceptable acid addition salt thereof or the N-oxide thereof, wherein
R is hydrogen, alkyl, alkenyl, alkynyl, cycloalkyl, cycloalkylalkyl or 2-hydroxyethyl;
R³ is CH₂—X—R′, wherein X is O, S, or NR″; R″ is hydrogen or alkyl; and
R′ is alkyl, alkenyl, alkynyl, cycloalkyl, cycloalkylalkyl, or —CO-alkyl;
R⁴ is phenyl, which may be substituted one or more times with substituents selected from the group consisting of halogen, CF₃, CN, alkoxy, cycloalkoxy alkyl, cycloalkyl, alkenyl, alkynyl, amino, nitro, and heteroaryl, and aryl; 3,4-methylenedioxyphenyl;
benzyl, which may be substituted one or more times with substituents selected from the group consisting of halogen, CF₃, CN, alkoxy, cycloalkoxy alkyl, cycloalkyl, alkenyl, alkynyl, amino, nitro, and heteroaryl, and aryl;
heteroaryl, which may be substituted one or more times with substituents selected from the group consisting of halogen, CF₃, CN, alkoxy, cycloalkoxy alkyl, cycloalkyl, alkenyl, alkynyl, amino, nitro, and heteroaryl, and aryl; or
naphtyl, which may be substituted one or more times with substituents selected from the group consisting of halogen, CF₃, CN, alkoxy, cycloalkoxy alkyl, cycloalkyl, alkenyl, alkynyl, amino, nitro, and heteroaryl, and aryl.

6. The method according to claim 1, wherein said monoamine neurotransmitter re-uptake inhibitor comprising a 2,3-disubstituted tropane moiety is a compound of formula (II) 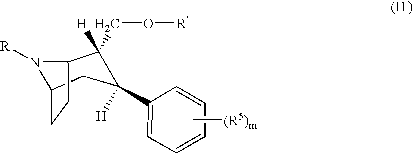
wherein
R represents a hydrogen atom or a C₁₋₆ alkyl group;
R⁵ represents a halogen atom or a CF₃ or cyano group;
R′ represents a hydrogen atom or a C₁₋₆ alkyl or C₃₋₆-cycloalkyl-C₁₋₃-alkyl group; and
m is 0 or an integer from 1 to 3;
or a tautomer, a pharmaceutically acceptable acid addition salt, or N-oxide thereof.

7. The method according to claim 1, wherein said monoamine neurotransmitter re-uptake inhibitor comprising a 2,3-disubstituted tropane moiety is the compound of formula (IA) or (IB) 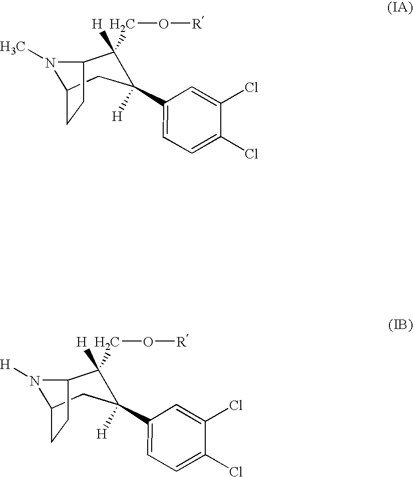
wherein R′ is methyl, ethyl or propyl or a pharmaceutically acceptable acid addition salt or N-oxide thereof.

8. The method according to claim 1, wherein the dosage amount of said monoamine neurotransmitter re-uptake inhibitor comprising a 2,3-disubstituted tropane moiety is from 0.05 to 10 mg per administration.

9. The method of claim 1 further comprising the step of administering a dopamine D₁₋, D₂₋, D₃₋ or D₄₋ agonists or a pharmaceutically acceptable salt thereof in a combined form with the monoamine neurotransmitter re-uptake inhibitor comprising a 2,3-disubstituted tropane moiety, or separately from the monoamine neurotransmitter re-uptake inhibitor comprising a 2,3-disubstituted tropane moiety, or separately and sequentially with the monoamine neurotransmitter re-uptake inhibitor comprising a 2,3-disubstituted tropane moiety.

10. The method according to claim 9, wherein the said dopamine D₁₋, D₂₋, D₃₋ or D₄₋ agonist is selected from the group consisting of adrogolide, A-86929, Rotigotine, NeurVex, nolomirole, pramipexole, talipexol, CHF 1512, (−)-stepholidine, DAR-201, diacrin/Genzyme, bromocriptine, bupropion, LEK-8829, BAM-1110, AIT-203, terguride, aripiprazole, OPC-4392, GMC-1111, PD-148903, apomorphine HCl, PD-89211, PD-158771, cabergoline, sumanirole, PNU-14277E, POL-255, dihydrexidine, GBR-12783, quinagolide HCl, (R)-bupropion, S-32504, S-33592, SKF-80723, SKF-83959, fenoldopam, Flibanserin, ropinirole, SKF-82958, SKF-77434, DU 127090, SLV-308, SLV 318, NeuroCRIB, SP-1037C, spheramine, gallotrank, preclamol, DAB-452, YM-435, BP-897, ProSavin, etilevodopa, P63, A 68930, A 77636, alaptide, alentemol, CI 1007; PD 143188, BLSI, JA 116a; JA 116, melevodopa; levodopa methyl; CHF 1301; NSC 295453; levomet, MR 708, PD 128483, RD 211, SKF 38393, SKF 81297, U 86170F, U 91356A, WAY 124486, Z 15040, silbutramine, orlistat, amfepramon-HCl, and ephedrine.

11. (canceled)
